(19) United States
(12) Patent Application Publication
(10) Pub. No.: US 2018/0008029 A1
(43) Pub. Date: Jan. 11, 2018

(54) SHEET-TYPE BEAUTY PACK

(71) Applicant: FANIPINKOREA CO., LTD., Cheongju-si, Chungcheongbuk-do (KR)

(72) Inventor: Hoi-Ki KIM, Cheongju-si (KR)

(73) Assignee: FANIPINKOREA CO., LTD., Cheongju-si, Chungcheongbuk-do (KR)

(21) Appl. No.: 15/532,727

(22) PCT Filed: Jun. 18, 2015
(86) PCT No.: PCT/KR2015/006163

§ 371 Date: Jun. 2, 2017

(30) Foreign Application Priority Data
Dec. 5, 2014 (KR) 10-2014-0173637

(51) Int. Cl. A45D 44/00; A61K 8/02; A61K 8/96; B32B 5/02; A45D 44/22; A61Q 19/00; B32B 7/06

(52) U.S. Cl. CPC A45D 44/002; A61Q 19/00; A61K 8/0212; B32B 7/06; B32B 5/022; A45D 44/22; A61K 8/965; A61K 2800/87; B32B 2555/00

(57) ABSTRACT

The present invention provides a sheet-type beauty pack having a convenient use and expected to provide moisture and nutrients to the skin of the face and the like, has a sufficient far infrared ray effect due to a natural material containing minerals, removes impurities, and has a wrinkle alleviation effect due to skin lifting, by applying a beauty pack, which uses a cosmetic material of a natural material containing minerals, in a paste state to a sheet such as nonwoven fabric. The sheet-type beauty pack comprises: a first release paper; a nonwoven fabric of which one surface is laminated with the first release paper and of which the other surface is coated with a cosmetic material in the paste state; and a second release paper laminated on the other surface, coated with the cosmetic material, of the nonwoven fabric.

### TECHNICAL FIELD

Embodiments of the present disclosure relate to a beauty pack, and more particularly, to a sheet-type beauty pack in which a natural raw material containing minerals is applied to a sheet such as a nonwoven fabric in a paste state.

### BACKGROUND ART

Recently, various cosmetic packs using natural raw materials containing minerals such as loess, mud, volcanic ash, and the like, have been developed.

In other words, it is known that loess is a fine porous material, has excellent adsorption property, contains various minerals, and radiates far-infrared rays. It is also known that, when loess is applied to the skin, cell tissues are activated by far-infrared rays to promote blood circulation and waste discharge, and foreign substances or waste placed deep inside pores of the skin are absorbed and removed by adsorption of the loess so that effects of whitening, moisturizing, detoxification, and the like can be obtained.

In addition, it is known that mud emits a large amount of far-infrared rays and is rich in various inorganic substances and vitamins, and thus mud has excellent effects such as removal of spots, freckles, and blemishes, promotion of skin regeneration, prevention of fine wrinkles, reduction of pores, removal of waste, and the like.

In addition, it is known that volcanic ash promotes metabolism by emitting a large amount of negative ions and far-infrared rays to the skin, and thus volcanic ash has effects of eliminating fungi and the like on the skin to alleviate skin troubles, alleviating wrinkles of the skin, preventing aging of the skin, and preventing and improving skin damage.

Accordingly, various cosmetic packs including at least one natural raw material containing such minerals have been developed. For example, Korean Patent Registration No. 873,563 (hereinafter, referred to as “Patent Document 1”) discloses a mixture of loess and charcoal powder formulated into cream or gel form, and Korean Patent Registration No. 787,105 (hereinafter, referred to as “Patent Document 2”) discloses a beauty massage loess pack comprising loess and sea tidal mud as main ingredients and a manufacturing method thereof.

As in Patent Documents 1 and 2, a cosmetic pack using a natural raw material containing minerals as a main ingredient is manufactured by formulating a natural raw material containing minerals in a paste state and putting the formulated material into tubes or containers. Here, a user applies a pack in the paste state within a tube or a container to the skin, such as his or her face, removes the pack, and washes the skin after a predetermined time has elapsed.

It is known that, when a cosmetic material comprising the natural raw material containing minerals in the paste state is applied directly to the skin such as a face, a wrinkle alleviation effect may be obtained by removing waste of the skin, moisturizing the skin, lifting the skin, and the like. However, it is not easy to uniformly apply the cosmetic material in the paste state to the skin such as the face, and it is difficult to adjust an amount of the cosmetic material to be used, resulting in a waste of a massage cosmetic material so that it is not only inconvenient to use the cosmetic material but also troublesome in treatment of a residue after using the pack.

Accordingly, various attempts have been made to improve convenience by applying a cosmetic pack composition using a natural raw material containing minerals as a main ingredient to a sheet such as a nonwoven fabric.

For example, Korean Patent Laid-Open Publication No. 2005-0067110 (hereinafter, referred to as “Patent Document 3”) discloses a method for manufacturing a gel-type pack in which loess gelled by pouring a gel formed by suspending loess among natural raw materials onto a nonwoven fabric surface is added to a gel-type pack. When a temperature of a cosmetic material gelled in this way rises (i.e., at human body temperature), the gelled cosmetic material is applied to the skin while the gel is melted, and the application is effective for moisturizing or nourishing the skin such as the face by using useful ingredients of loess and is also effective for removing waste matter when the loess is packed by applying conventional loess to the skin such as the face. However, when the conventional loess is made into a paste state and put directly on the skin such as the face, a wrinkle alleviation effect caused by waste removal or skin lifting is insignificant, and since the gel is melted when a temperature rises, there is a problem that the loess flows down along with water containing the loess during storage or during use.

In addition, Korean Patent Laid-Open Publication No. 2007-0081813 (hereinafter, referred to as “Patent Document 4”) discloses a loess face mask pack in which a fabric is passed through a container containing a lightly kneaded loess dough for 2 to 5 seconds so that loess is absorbed into the fabric, and then liquid is squeezed out of the fabric and the fabric is dried at a high temperature. The loess face mask pack is obtained by adsorbing a loess material onto a nonwoven fabric and then drying the loess material in the same manner as that of the nonwoven fabric and is dyed by being immersed in a loess solution. When the loess face mask pack is used, a cosmetic toner is first sprayed onto the skin such as the face, and then the mask pack is used in close contact with the skin. Here, it is effective to moisturize or nourish the skin such as the face by using useful ingredients of loess as in Patent Document 3, but it is difficult to expect the wrinkle alleviation effect caused by waste removal or skin lifting when the conventional loess is made into the paste state and put directly on the skin such as the face.

As described above, conventionally, when a cosmetic material using natural raw materials containing minerals such as loess, mud, volcanic ash, and the like is directly applied to a sheet such as a nonwoven fabric in the paste state, the cosmetic material flows down due to water contained in the cosmetic material so that it is difficult to store and use the cosmetic material, resulting in difficulties in commercialization of the cosmetic material.
Patent Document 1: Korean Patent Registration No. 873,563
Patent Document 2: Korean Patent Registration No. 787,105
Patent Document 3: Korean Patent Laid-Open Publication No. 2005-0067110
Patent Document 4: Korean Patent Laid-Open Publication No. 2007-0081813

### DISCLOSURE

### Technical Problem

The present disclosure is directed to providing a sheet-type beauty pack in which a cosmetic material of a natural raw material containing minerals is applied to a sheet such as a nonwoven fabric in a paste state so that the beauty pack may moisturize or nourish the skin such as a face, have ease of use, and be expected to have a sufficient far-infrared ray effect due to the natural raw material containing minerals and a wrinkle alleviation effect caused by waste removal or skin lifting.

### Technical Solution

To address the above-described problems, the present disclosure provides a sheet-type beauty pack including: a first release paper; a nonwoven fabric of which one surface is laminated with the first release paper and the other surface is coated with a cosmetic material in a paste state; and a second release paper that is laminated on the other surface, which is coated with the cosmetic material, of the nonwoven fabric, wherein the cosmetic material includes 35 to 45 parts by weight of a natural raw material containing minerals, 3 to 6 parts by weight of a dispersant, 0.1 to 8 parts by weight of a thickener, and 45 to 55 parts by weight of purified water, and the natural raw material containing minerals includes at least one of powdery loess, powdery mud, and powdery volcanic ash.

Also, the nonwoven fabric may be a long-fiber nonwoven fabric.

Also, the long-fiber nonwoven fabric may be any one of a long-fiber nonwoven fabric using silk, a long-fiber nonwoven fabric in which cellulose is melted and continuously spun, and a long-fiber nonwoven fabric in which a polymer raw material is changed into a liquid to be continuously spun.

Also, the nonwoven fabric may be a nonwoven fabric having a three-layer structure in which a melt-blown nonwoven fabric made of superfine fibers is used as an intermediate layer and a spun-bonded long-fiber nonwoven fiber is stacked on both of the surfaces of the nonwoven fabric.

Also, a surface of the nonwoven fabric may be subjected to a hydrophilization treatment using a hydrophilization agent after being subjected to a surface treatment by a corona discharge so that hydrophilicity and an adhesive force of the surface are improved.

### Advantageous Effects

According to the present invention, a sheet-type beauty pack in which a cosmetic material of a natural raw material containing minerals is applied to a sheet such as a long-fiber nonwoven fabric in a paste state so that the beauty pack may moisturize or nourish the skin such as a face, have ease of use, and be expected to have a sufficient far-infrared ray effect due to the natural raw material containing minerals and to have a wrinkle alleviation effect caused by waste removal or skin lifting.

### DESCRIPTION OF DRAWINGS

FIG. 1 is a view showing a structure of a sheet-type beauty pack according to an embodiment of the present invention.

FIG. 2 is photographs for comparing surface states of a long-fiber nonwoven fabric and a short-fiber nonwoven fabric, wherein (a) is a photograph showing the surface of the long-fiber nonwoven fabric and (b) is a photograph showing the surface of the short-fiber nonwoven fabric.

FIG. 3 is photographs for comparing sheet states after use according to a type of nonwoven fabric of a sheet-type beauty pack according to an embodiment of the present invention, wherein (a) is a photograph showing an example by a long-fiber nonwoven fabric and (b) is a photograph showing an example by a short-fiber nonwoven fabric.

FIG. 4 is a view showing a cross-section of a three-layer nonwoven fabric in a sheet-type beauty pack according to an embodiment of the present invention.

FIG. 5 is a view showing an example of a manufacturing process of a sheet-type beauty pack according to an embodiment of the present invention.

### MODES OF THE INVENTION

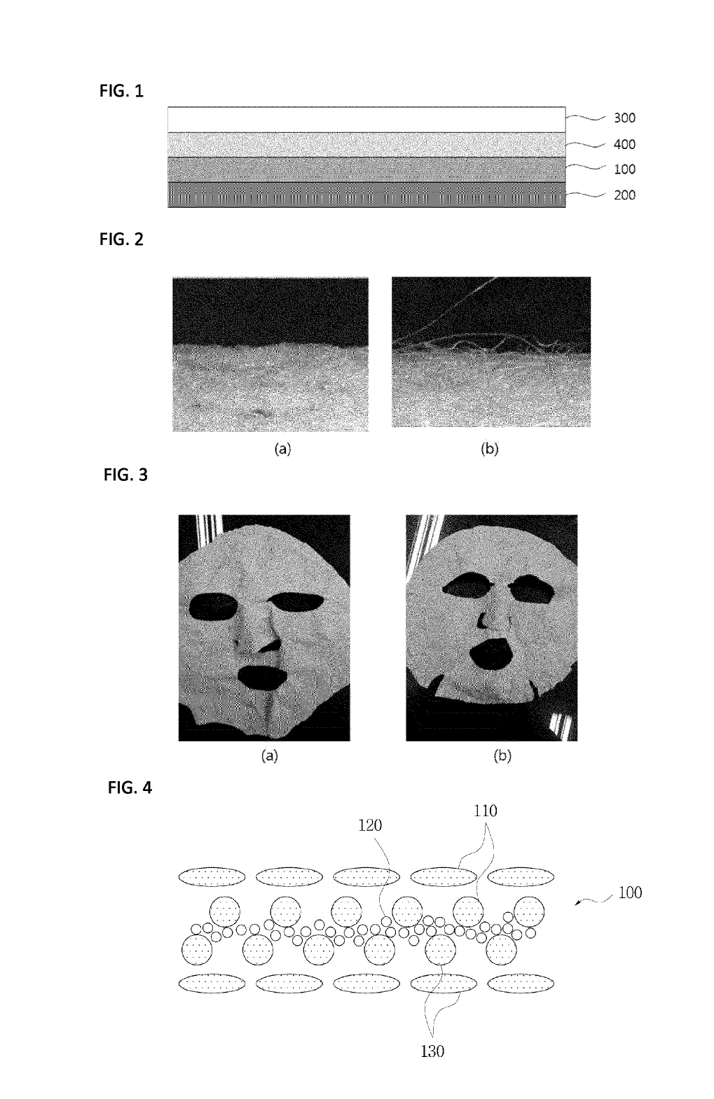

Hereinafter, embodiments of the present invention will be described in detail with reference to FIGS. 1 to 5.

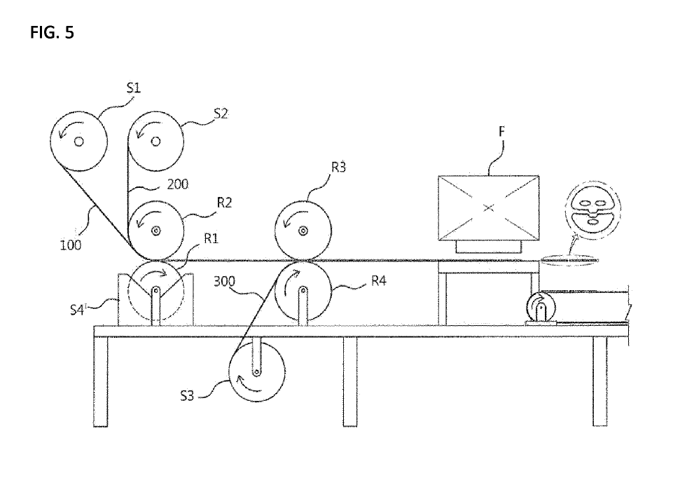

FIG. 1 is a view showing a structure of a sheet-type beauty pack according to an embodiment of the present invention, FIG. 2 is photographs for comparing surface states of a long-fiber nonwoven fabric and a short-fiber nonwoven fabric, wherein (a) is a photograph showing the surface of the long-fiber nonwoven fabric and (b) is a photograph showing the surface of the short-fiber nonwoven fabric, FIG. 3 is photographs for comparing sheet states after use according to a type of a nonwoven fabric of a sheet-type beauty pack according to an embodiment of the present invention, wherein (a) is a photograph showing an example by a long-fiber nonwoven fabric and (b) is a photograph showing an example by a short-fiber nonwoven fabric, FIG. 4 is a view showing a cross-section of a three-layer nonwoven fabric in a sheet-type beauty pack according to an embodiment of the present invention, and FIG. 5 is a view showing an example of a manufacturing process of a sheet-type beauty pack according to an embodiment of the present invention.

First, as shown in FIG. 1, a sheet-type beauty pack (hereinafter, referred to as “beauty pack”) includes a first release paper 200, a nonwoven fabric 100 of which one surface is laminated with the first release paper 200 and the other surface is coated with a cosmetic material 400 in a paste state, and a second release paper 300 laminated on the other surface, which is coated with the cosmetic material 400, of the nonwoven fabric 100, as a three-layer stacked structure.

The cosmetic material 400 applied in the paste state on the other surface of the nonwoven fabric 100, of which one surface is laminated with the first release paper 200, penetrates into pores of the nonwoven fabric by being press-fitted in a laminating process of the first release paper 200 and the second release paper 300, and is spread evenly on the nonwoven fabric. At this time, the cosmetic material needs to be appropriately restricted in fluidity so that the cosmetic material is applied in the paste state so as not to flow.

Therefore, a specific technical means for restricting the fluidity of the cosmetic material in the paste state will be described in detail below in view of each of the cosmetic material 400, the nonwoven fabric 100, and the release papers 200 and 300.

[Cosmetic Material 400]

In the present invention, a composition ratio and application amount of the cosmetic material 400 should be carefully designed in consideration of physical properties (e.g., spreadability on the nonwoven fabric, an adhesive force, viscosity, releasability of the release paper, etc.) required for applying the cosmetic material in the paste state to the nonwoven fabric as well as functionality as a beauty pack.

Accordingly, in the present invention, the cosmetic material 400 includes 30 to 45 parts by weight of a natural raw material containing powdery minerals, 3 to 6 parts by weight of a dispersant, 0.1 to 8 parts by weight of a thickener, 45 to 55 parts by weight of purified water, and appropriate amounts of other additives such as spices or vitamins.

The natural raw material containing minerals includes at least one of powdery loess, powdery sea tidal mud (hereinafter, referred to as “mud”), and powdery volcanic ash, and such loess, mud, or volcanic ash is known to contain a large amount of inorganic nutrients such as minerals and emit far-infrared rays.

Far-infrared rays penetrate deeply into the skin of a human body to generate heat. Here, it is known that this heat effect helps to eliminate germs that cause various diseases, enlarges capillaries to help blood circulation and cell tissue formation, and activates cell tissues to prevent various adult diseases, for example, preventing aging, promoting metabolism, relieving chronic fatigue, and the like.

Meanwhile, the natural raw material containing minerals further includes charcoal powder in addition to at least one of the powdery loess, the powdery mud, and the powdery volcanic ash, and the charcoal powder is known to have the far-infrared ray effect as well as an effect of eliminating even sebum of fine pores of the skin because the charcoal powder has a porous structure so that it is excellent in adsorption power.

Content of the natural raw material containing such minerals is preferably 30 to 45 parts by weight.

That is, when the content of the natural raw material is 30 parts by weight or less, an amount of purified water, which will be described later, may be relatively increased. When the amount of purified water is increased, a state of the entire cosmetic material 400 is diluted to cause an increase in the fluidity thereof, and when an amount of the thickener or other additives is increased in order to restrict the fluidity, it is difficult to fully utilize functions and effects of the natural raw material in addition to increasing cost.

In addition, when the content of the natural raw material is 45 parts by weight or greater, the amount of contained purified water is relatively reduced so that the viscosity of the cosmetic material 400 is increased in addition to the spreadability of the cosmetic material 400 on the nonwoven fabric 100 being deteriorated, so that releasability of the release papers 200 and 300 may be deteriorated and an unpleasant feeling due to stickiness may occur during use.

Meanwhile, since the loess, the mud, or the volcanic ash is not easy to disperse in water, the cosmetic material 400 includes 3 to 6 parts by weight of the dispersant. The dispersant is not particularly limited as an additive used for uniformly dispersing liquids or solids which do not mix with each other well in a liquid, but a nonionic surfactant such as polysorbate 80 may be preferably used.

In addition, the thickener is used to achieve an appropriate viscosity to restrict the fluidity so that the cosmetic material 400 may be prevented from dripping in a state in which the cosmetic material 400 is attached to the nonwoven fabric 100. Here, the thickener is not particularly limited, but xanthan gum or the like may be used, and the cosmetic material 400 includes 0.1 to 8 parts by weight of the thickener. When the viscosity of a natural raw material itself such as loess and mud is large, a separate thickener may be unnecessary, but the natural raw material itself may be appropriately added in the range of 0.1 to 8 parts by weight so that the viscosity is not uniform. However, when the content of the thickener is 8 parts by weight or greater, the viscosity of the cosmetic material 400 becomes high, and thus the cosmetic material 400 may excessively come out of the release papers 200 and 300 when the release papers 200 and 300 are removed or an unpleasant feeling such as stickiness may be generated when the cosmetic material 400, whose viscosity is high, is used on the skin such as the face.

Generally, a content ratio of purified water contained in a cosmetic toner or a beauty pack is not considered important, but in the present invention, the content ratio of the purified water is important in that the fluidity of the cosmetic material 400 should be appropriately restricted to the paste state. That is, when the amount of the purified water contained as described above is large, there is a problem in that the cosmetic material 400 in the paste state becomes excessively diluted and easily drips. When the amount of purified water contained in the cosmetic material 400 is small, there are problems in that the spreadability of the cosmetic material 400 on the nonwoven fabric 100 and the releasability of the release papers 200 and 300 are deteriorated and an unpleasant feeling due to stickiness is generated during use of the cosmetic material 400.

In addition, in a state in which the beauty pack according to the present invention is attached to the skin such as the face, useful ingredients of the natural raw material containing minerals may act on the skin while moisture contained in the cosmetic material 400 is slowly evaporated (that is, dried), and a wrinkle alleviation effect caused by waste removal or skin lifting of the skin may be expected. In other words, it is preferable that a time required for the cosmetic material 400 to be completely dried after attaching the beauty pack to the skin is at least 20 minutes or more. That is, useful ingredients of the natural raw material may act on the skin while the cosmetic material 400 is slowly evaporated for 20 minutes or more, and the wrinkle alleviation effect may be exhibited.

The content of purified water is preferably 45 to 55 parts by weight in consideration of the above-described functional and physical properties of the cosmetic material 400.

[Nonwoven Fabric 100]

The nonwoven fabric 100 acts as a support for allowing the cosmetic material 400 to be attached in the paste state, and needs to allow the cosmetic material 400 to be attached so that the cosmetic material 400 may be prevented from dripping from the nonwoven fabric 100.

Such nonwoven fabric is classified into a long-fiber nonwoven fabric and a short-fiber nonwoven fabric. Here, as to the short-fiber nonwoven fabric mainly used in a conventional sheet-type pack, short staples, each usually having a length of 2.5 to 3.8 cm, are spun to form a sheet so that a basis weight (a weight per unit area) is not constant, a surface thereof is not flat, and fluff is formed (see, (a) of FIG. 2). Conversely, as to the long-fiber nonwoven fabric, spun filaments are produced in a single continuous process so that a deviation of the basis weight is not greater than that of the single-fiber nonwoven fabric, a surface thereof is flat, and no fluff is formed (see, (b) of FIG. 2).

Accordingly, when the cosmetic material 400 in the paste state is applied to the short-fiber nonwoven fabric as in the present invention, not only is the spreadability deteriorated but an adhesive force for attaching the cosmetic material 400 applied to the nonwoven fabric 100 is also deteriorated.

Meanwhile, since the long-fiber nonwoven fabric has a small deviation of the basis weight, has the flat surface, and has no fluff, unlike the short-fiber nonwoven fabric, not only is excellent spreadability exhibited but an excellent adhesive force for attaching the cosmetic material 400 applied to the nonwoven fabric 100 is also exhibited even when the cosmetic material 400 in the paste state is applied to the long-fiber nonwoven fabric.

In addition, in a case in which the beauty pack according to the present invention is manufactured and used using actual long-fiber nonwoven fabric and short-fiber nonwoven fabric, when the long-fiber nonwoven fabric is used as shown in (a) of FIG. 3, it can be seen that the beauty pack is attached to the skin and slowly dried, and then a surface of the dried beauty pack appears smooth. However, when the short-fiber nonwoven fabric is used as shown in (b) of FIG. 3, it can be seen that the beauty pack is attached to the skin and slowly dried, and then a lot of fine wrinkles are formed on a surface (especially a jaw portion) of the dried beauty pack. That is, when the beauty pack according to the present invention is attached to the skin, the cosmetic material of the beauty pack is slowly dried to shrink and lift the skin. At this time, when the cosmetic material 400 is applied to the short-fiber nonwoven fabric, it can be seen that a sufficient lifting effect is not exhibited due to the formation of fine wrinkles.

Accordingly, it is preferable that the nonwoven fabric 100 used in the beauty pack according to the present invention be the long-fiber nonwoven fabric.

As the long-fiber nonwoven fabric, a nonwoven fabric in which natural long fibers such as silk are spun to form a web (a state in which the fibers are repeatedly matched) may be used, or a nonwoven fabric by a spun bond method in which a polymer raw material, such as polyester, nylon, or polypropylene, or cellulose is changed into a liquid to be continuously spin-produced may be used. As to the nonwoven fabric by the spun bond method, filaments spun from an extruder are stretched and then stacked to form the web. Here, the nonwoven fabric by the spun bond method has practical strength due to a long fiber length thereof, has excellent air permeability, and has properties such as water repellency specific to the fibers because the fibers are sheeted as they are without performing an emulsion treatment or the like thereon.

Meanwhile, cellulose is the most abundant organic compound among organic compounds produced in nature and has a very strong hydrogen bond due to a hydroxyl group thereof, and thus cellulose itself has no thermoplasticity. However, a cellulose hydroxyl group may be blocked and derived to impart thermoplasticity to the cellulose. In this manner, the long-fiber nonwoven fabric may be obtained by melting and spinning cellulose to which thermoplasticity is imparted.

Accordingly, since the nonwoven fabric 100 of the beauty pack is directly brought into contact with the skin, a long-fiber nonwoven fabric using silk as a natural raw material or a long-fiber nonwoven fabric obtained by melting and spinning cellulose is preferably used. In particular, a long-fiber nonwoven fabric obtained by melting and spinning cellulose obtained from a cotton linter (cotton fleece attached to cottonseed) is more preferably used because it is economically excellent.

In addition, as shown in FIG. 4, a nonwoven fabric having a three-layer structure which includes a thermoplastic long fiber layer 110, a thermoplastic microfine fiber layer 120, and a thermoplastic long fiber layer 130 may be used as the nonwoven fabric 100. At this time, a total thickness of the three-layered nonwoven fabric is preferably 15 to 300 μm.

By using the thermoplastic microfine fiber layer 120 as an intermediate layer in this way, penetration of the cosmetic material applied to the nonwoven fabric into even a back surface (that is, a surface to be laminated with the first release paper 120) may be minimized, and an amount of the applied cosmetic material may be economically reduced.

At this time, the thermoplastic long fiber layers 110 and 130 constituting surface layers of the nonwoven fabric having the three-layer structure are long-fiber nonwoven fabrics produced in such a manner that a thermoplastic resin melted by the above-described spun bond method is extruded from a circular capillary-shaped spinneret so that the extruded thermoplastic resin is melted and spun to be continuously stacked. The thermoplastic microfine fiber layer 120 constituting the intermediate layer is a nonwoven fabric by a melt blown method.

Through the melt blown method, a microfine fiber sheet may be obtained in such a manner that fibers of a thermoplastic resin such as polypropylene are stretched and opened by introducing high-temperature and high-pressure air streams into an outlet of a spinning nozzle, and then stacked.

In addition, the nonwoven fabric 100 may improve an adhesive force of the cosmetic material 400 by applying a hydrophilization treatment to a surface to which the cosmetic material 400 is applied.

At this time, the hydrophilization treatment of the nonwoven fabric 100 may be performed by a known method such as a dipping method, a spraying method, or a coating method by using a diluted hydrophilic agent solution. Here, prior to the hydrophilization treatment, a surface treatment such as a corona treatment is preferably performed from the standpoint of improving the degree of hydrophilization. That is, when the surface of the nonwoven fabric 100 is irradiated by a corona discharge, hydrophilicity increases and the adhesive force is further improved.

[Release Papers 200 and 300]

The first release paper 200 and the second release paper 300 are laminated on both side surfaces of the nonwoven fabric 100 to which the cosmetic material 400 is applied so that the cosmetic material 400 applied to the nonwoven fabric 100 may be protected and the cosmetic material 400 applied and attached to the nonwoven fabric 100 may be prevented from dripping.

As in a manufacturing process described below, the nonwoven fabric 100 is laminated with the first release paper 200, the cosmetic material 400 is applied to the nonwoven fabric 100, and then the second release paper 300 is laminated on the nonwoven fabric 100. In a laminating process of the release papers, the cosmetic material 400 applied to the nonwoven fabric 100 is impregnated into the nonwoven fabric 100 through pores of the nonwoven fabric 100, and the first release paper 200 and the second release paper 300 are attached due to the viscosity of the cosmetic material 400.

The first release paper 200 and the second release paper 300 laminated in this manner are removed to allow a user to use the beauty pack, and at this time, it is necessary to minimize the amount of the cosmetic material 400 that is attached to and comes out of the removed first release paper 200 and second release paper 300 (especially, the second release paper 300).

Therefore, it is preferable that a non-polar polymer having low surface tension, for example, polyethylene (PE), is used as the first release paper 200 and the second release paper 300. In particular, it is preferable that at least the second release paper 300 is made of polyethylene having high transparency so that the cosmetic material applied to the nonwoven fabric 100 can be visually confirmed.

As described above, a composition and content of the beauty pack according to the present invention may be carefully designed to be in the paste state so that the cosmetic material 400 in the paste state, which is applied to the nonwoven fabric 100, may be prevented from dripping when it is stored, transported, or even used by means of the cosmetic material 400 whose fluidity is appropriately controlled, the nonwoven fabric 100 with the improved spreadability on and adhesive force of the cosmetic material 400, are improved, and the first release paper 200 and second release paper 300, which are attached to the cosmetic material 400 applied to the nonwoven fabric 100 and restrict the fluidity of the cosmetic material 400.

Hereinafter, an example of a manufacturing process of a beauty pack according to the present invention will be described with reference to FIG. 5.

First, the first release paper 200 is laminated on one surface of the nonwoven fabric 100 while the nonwoven fabric 100 supplied from a nonwoven fabric supply unit S1 and the first release paper 200 supplied from a first release paper supply unit S2 pass through a pair of laminated rollers R1 and R2. At this time, the cosmetic material 400 inside a cosmetic material supply unit S4 is put on the laminated roller R1 on a side of the nonwoven fabric 100 and applied to the nonwoven fabric 100, and the nonwoven fabric 100 and the first release paper 200 are pressed by the pair of laminated rollers R1 and R2 and the cosmetic material 400 is impregnated into the nonwoven fabric 100 at the same time.

Next, the second release paper 300 supplied from a second release paper supply unit S3 is supplied to the cosmetic material 400 applied on the nonwoven fabric 100 laminated with the first release paper 200 in this manner, and the second release paper 300 is laminated on a surface on which the cosmetic material of the nonwoven fabric 100 is applied while passing through a pair of laminated rollers R3 and R4, and the cosmetic material 400 applied to the nonwoven fabric 100 is uniformly attached to the nonwoven fabric by pressure of the rollers again while simultaneously filling pores of the nonwoven fabric 100.

Such a three-laminated beauty pack including the first release paper 200, the nonwoven fabric 100, and the second release paper 300 is wrapped after being cut into a human face shape or the like in a foaming unit F.

### INDUSTRIAL APPLICABILITY

The disclosed embodiment relates to a beauty pack which is industrially applicable.

1. A sheet-type beauty pack comprising:
a first release paper;
a nonwoven fabric of which one surface is laminated with the first release paper and the other surface is coated with a cosmetic material in a paste state; and
a second release paper that is laminated on the other surface, which is coated with the cosmetic material, of the nonwoven fabric,
wherein the cosmetic material includes 35 to 45 parts by weight of a natural raw material containing minerals, 3 to 6 parts by weight of a dispersant, 0.1 to 8 parts by weight of a thickener, and 45 to 55 parts by weight of purified water, and the natural raw material containing minerals includes at least one of powdery loess, powdery mud, and powdery volcanic ash.

2. The sheet-type beauty pack of claim 1, wherein the nonwoven fabric is a long-fiber nonwoven fabric.

3. The sheet-type beauty pack of claim 2, wherein the long-fiber nonwoven fabric is any one of a long-fiber nonwoven fabric using silk, a long-fiber nonwoven fabric in which cellulose is melted and continuously spun, and a long-fiber nonwoven fabric in which a polymer raw material is changed into a liquid to be continuously spun.

4. The sheet-type beauty pack of claim 1, wherein the nonwoven fabric is a nonwoven fabric having a three-layer structure in which a melt-blown nonwoven fabric made of superfine fibers is used as an intermediate layer and a spun-bonded long-fiber nonwoven fiber is stacked on both of the surfaces of the nonwoven fabric.

5. The sheet-type beauty pack of claim 1, wherein a surface of the nonwoven fabric is subjected to a hydrophilization treatment using a hydrophilization agent after being subjected to a surface treatment by a corona discharge so that hydrophilicity and an adhesive force of the surface are improved.

6. The sheet-type beauty pack of claim 2, wherein a surface of the nonwoven fabric is subjected to a hydrophilization treatment using a hydrophilization agent after being subjected to a surface treatment by a corona discharge so that hydrophilicity and an adhesive force of the surface are improved.

7. The sheet-type beauty pack of claim 3, wherein a surface of the nonwoven fabric is subjected to a hydrophilization treatment using a hydrophilization agent after being subjected to a surface treatment by a corona discharge so that hydrophilicity and an adhesive force of the surface are improved.

8. The sheet-type beauty pack of claim 4, wherein a surface of the nonwoven fabric is subjected to a hydrophilization treatment using a hydrophilization agent after being subjected to a surface treatment by a corona discharge so that hydrophilicity and an adhesive force of the surface are improved.
